(19) United States
(12) Patent Application Publication
(10) Pub. No.: US 2005/0004649 A1
(43) Pub. Date: Jan. 6, 2005

(54) Radiopaque balloon

(75) Inventors: Bhavesh Mistry, Cupertino, CA (US); Jeffrey A. Sarge, Fremont, CA (US); Frank A. Musbach, Pleasanton, CA (US)

Correspondence Address: VIDAS, ARRETT & STEINKRAUS, P.A., 6109 BLUE CIRCLE DRIVE, SUITE 2000, MINNETONKA, MN, 55343-9185, US

(73) Assignee: SCIMED LIFE SYSTEMS, INC., Maple Grove, MN (US)

(21) Appl. No.: 10/897,868

(22) Filed: Jul. 23, 2004

Related U.S. Application Data
(62) Division of application No. 09/892,048, filed on Jun. 26, 2001, now abandoned.

(51) Int. Cl. A61F 2/06

(52) U.S. Cl. 623/1.11

(57) ABSTRACT

A stent delivery device which includes a balloon having at least one radiopaque region or portion.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

The present invention is directed to a balloon catheter having at least one radiopaque region. Specifically, the present invention is directed to a stent delivery catheter in which marker bands are included on the balloon instead of on the catheter.

2. Description of the Related Art

Stents for transluminal implantation are well known. They are generally comprised of metallic supports which are inserted into a part of the human body such as bile ducts, the urinary system, the digestive tube and notably by percutaneous route inside the blood vessels, usually the arteries in which case they are typically termed vascular stents. Stents are usually generally cylindrical and are constructed and arranged to expand radially once in position within the body. They are usually inserted while they have a first relatively small diameter and implanted in a desired area, for example inside a vessel, then the stent is expanded in situ until it reaches a second diameter larger than the first diameter.

A balloon associated with the catheter is usually used to provide the necessary interior radial force to the stent to cause it to expand radially. An example of a balloon expandable stent is shown in U.S. Pat. No. 4,733,665 to Palmaz, which issued Mar. 29, 1988, and discloses a number of stent configurations for implantation with the aid of a catheter. The catheter includes an arrangement wherein a balloon inside the stent is inflated to expand the stent by plastically deforming it, after positioning it within a blood vessel.

Marker bands have been placed on the inner shaft of the catheter on either side of the stent (which is mounted to the balloon) to mark the ends of the stent during delivery. However, placing the marker bands onto the inner shaft of the catheter requires a crimping step which can result in scraped devices due to misaligned marker bands.

U.S. Pat. No. 5,820,585 issued Oct. 13 1998 discloses an angiographic and arteriographic balloon catheter in which the proximal and distal ends of the balloon are marked with a radiopaque print ink. However, this references fails to appreciate the advantages of a balloon with radiopaque regions for use in stent delivery.

The entire content of all of the patents listed within the present patent application are incorporated herein by reference.

### BRIEF SUMMARY OF THE INVENTION

Applicants have eliminated the need for marker bands to be manually crimped onto the inner shaft of the catheter by etching defined positions onto the balloon itself to indicate where radiopaque ink material will be sprayed onto the balloon to function as marker bands on the balloon itself, rather than on the catheter inner shaft. This eliminates waste due to misaligned marker bands; reduces the manufacturing cost and reduces device profile.

Applicants have invented a balloon catheter which includes a balloon having at least one radiopaque portion. The radiopaque portion or region is created by spraying radiopaque ink onto the balloon where desired. Although any desired portion of the balloon could be made radiopaque, one embodiment of the invention is to create two radiopaque marker band regions on the balloon which mark the ends of the stents mounted on the balloon for delivery.

Additional details and/or embodiments of the invention are discussed below.

### BRIEF DESCRIPTION OF THE SEVERAL VIEWS OF THE DRAWING(S)

FIG. 1 is a side cross-sectional view of the inventive balloon catheter.

### DETAILED DESCRIPTION OF THE INVENTION

While this invention may be embodied in many different forms, there are shown in the drawings and described in detail herein specific embodiments of the invention. The present disclosure is an exemplification of the principles of the invention and is not intended to limit the invention to the particular embodiments illustrated.

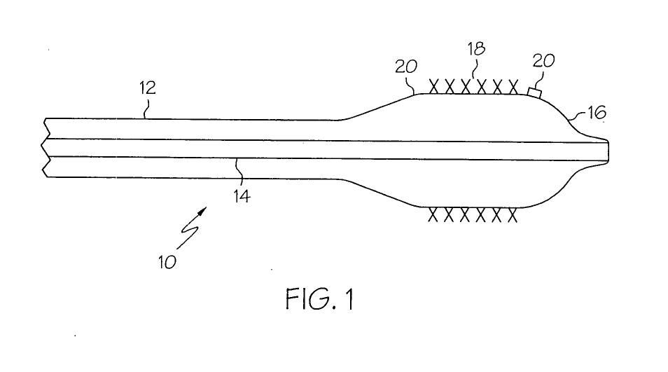

FIG. 1 shows a balloon catheter shown generally at 10, having an outer shaft 12 and inner shaft 14, the inner and outer shafts forming an inflation lumen. The proximal end of a balloon 16 is connected to outer shaft 10 and the distal end of balloon 16 is attached to the inner shaft 14. This balloon construction is well known in the art.

A stent 18 is mounted for delivery onto balloon 16, as is well known in the art.

Radiopaque coating or ink has been sprayed onto balloon 16 in concentric bands shown at 20 and these two radiopaque ink balloon regions or portions mark or indicate the distal and proximal ends of the stent. In at least one alternative embodiment, the balloon 16 may be marked by suspending radiopaque metallic powder in a resin/solvent mixture which is compatible with the material of the balloon 16. The mixture may then be painted or otherwise applied to the balloon surface, after which the solvent is allowed to flash off. The coating may be comprised of Radiopaque ink such as may be found in MICROFUSED™ radiopaque coatings available from Implant Sciences Corp. of Wakefield, Mass. Because the material of the balloon 16 typically will “expand” primarily by unfolding rather than by stretching, the radiopaque coating or ink will tend not to peel during balloon 16 expansion.

In order to locate the desired locations to apply the radiopaque ink, the balloon mold can be etched to make marks on the balloon circumference to indicate where the radiopaque ink should be applied.

Although radiopaque ink is preferred, radiopaque metal powder could be used when molding the balloon to create radiopaque regions.

Although only two radiopaque bands have been shown, it should be understood that any desired portion of the balloon could be made radiopaque using radiopaque ink or metal powder.

By placing the radiopaque marker bands onto the balloon 16 itself, marker bands can be eliminated from inner shaft 14. This eliminates a crimping step and potential waste due to marker band misalignment, making the manufacturing process cheaper.

This completes the description of the preferred and alternate embodiments of the invention. Those skilled in the art may recognize other equivalents to the specific embodiment described herein which equivalents are intended to be encompassed by the claims attached hereto.

1. A stent delivery catheter comprising:
a catheter having a distal end, the catheter including a balloon near its distal end;
a stent mounted for delivery onto the balloon,
the balloon including at least one radiopaque portion.

2. The stent delivery catheter of claim 1 wherein the radiopaque portion is made by applying radiopaque ink to the balloon.

3. The stent delivery catheter of claim 1 wherein the radiopaque portion is made by including radiopaque metal powder in the balloon material.

4. The stent delivery catheter of claim 1 wherein the at least one radiopaque portion comprises one or more radiopaque band portions.

5. The stent delivery catheter of claim 4 further wherein the stent has proximal and distal ends, and further wherein the two radiopaque band portions are positioned so that they mark the proximal and distal ends of the stent.

6. A method of making a stent delivery balloon comprising the steps of:
providing a balloon mold which is etched to make marks on the balloon;
molding the balloon using the provided balloon mold, and
applying radiopaque ink to the balloon portion defined by the marks made by the etched balloon mold.
